(19) United States
(12) Patent Application Publication
(10) Pub. No.: US 2024/0164699 A1
(43) Pub. Date: May 23, 2024

(54) DEVICE FOR PREDICTING THE WEARING-OFF CONDITIONS IN A PATIENT AFFECTED BY PARKINSON'S DISEASE

(71) Applicant: OFFXET S.R.L., Padova (IT)

(72) Inventors: Giovanni GENTILE, Scicli (IT); Arberto RUZZON, Stra (IT); Claudio MANCA, Padova (IT); Roberta DE MITRI, Padova (IT)

(21) Appl. No.: 18/282,552

(22) PCT Filed: Mar. 17, 2022
(86) PCT No.: PCT/IB2022/052423

§ 371 Date: Sep. 18, 2023

(30) Foreign Application Priority Data
Mar. 17, 2021 (IT) 102021000006362

(51) Int. Cl. A61B 5/00; A61B 5/0531

(52) U.S. Cl. CPC A61B 5/4082; A61B 5/0022; A61B 5/0531; A61B 5/681

(57) ABSTRACT

Device for predicting the wearing-off conditions in a patient affected by Parkinson's disease, which comprises an electrodermal activity sensor that detects the skin conductance of a patient and generates corresponding measurement signals; an electronic control unit, which receives the above measurement signals and calculates a physiological parameter representative of the skin conductance; a communication unit, which is connected to the electronic control unit and is actuatable by the latter to emit an off-notification signal. The electronic control unit comprises a processing module which detects, within a movable time window, an overall time variation of the physiological parameter indicative of an increase of the skin conductance, and compares this overall time variation with a specific threshold value, so as to control the communication unit to emit an off-notification signal which allows warning, ahead of time, the verification of a probable proximate wearing-off, thus allowing suitable operation.

### FIELD OF APPLICATION

The present invention regards a device for predicting the wearing-off conditions in a patient affected by Parkinson's disease.

The present device is inserted in the field of biomedical devices employable, in particular, in neurological field, for detecting specific clinical conditions (termed wearing-off conditions) in subjects affected by Parkinson's disease.

In particular, the present device is adapted to be worn by a patient and is intended to be advantageously used, both in hospital or convalescent environments (hospitals, nursing homes, etc.) and in home or civil environments, in order to predict, ahead of time, the onset of the wearing-off conditions in the patient and allow the use of suitable intervention measurements.

### STATE OF THE ART

In field of psychiatry or neurology, or generally of the clinical conditions of the central nervous system, there is the particular need to identify objective biomarkers with respect to the variation of the clinical state of the patient. Such requirement is particularly important both in the field of clinical trials, and in the therapeutic treatment applied to patients subjected to pathologies of the central nervous system.

In recent years, wearable electronic instruments have been developed that allow the detection of specific biomarkers (such as heart rate, motor conditions, respiratory rate, etc.) and the collection in digital format in order to allow the analysis of the detected data.

In particular, in the study and treatment of Parkinson's disease, it is of particular interest to identify biomarkers which allow executing the monitoring of the motor difficulties that such disease involves (such as for example tremors or motor block states) and due to the degeneration of the central nervous system.

However, in addition to the motor symptoms, there are also other very impacting symptoms which are also tied to the more or less correct response to a pharmacological treatment: they are expressed in a wide range of symptoms including cardiovascular dysfunctions, sweating, motor difficulty, pain, sleep disturbances, etcetera.

Even if therapeutic treatments (e.g. based on levodopa) have been developed which allow obtaining an initial symptomatic control, in the course of several years the patients often develop complications due to the response to the treatment whose effect wears off, as well as due to involuntary movements or dyskinesia induced by the drug. Specifically, a particular symptom to which the patients affected by Parkinson's disease are subjected is tied to the arrival of the so-called wearing-off conditions.

Such wearing-off conditions are clinical steps, which generally arise following complications in the advanced phase of the disease, in which the patient, even in the presence of a well-calibrated pharmacological therapy, is subjected to generalized motor blocks of relatively long duration, in which symptoms such as tremors, rigidity or slowness in movements, difficulty in starting movements, involuntary movements, dyskinesia, dystonia can be manifested, which can be accompanied by symptoms of psychosomatic character such as anxiety, fatigue, change of humor, difficulty in thought, agitation, etcetera.

At the present state of the art, the monitoring and the study of the aforesaid wearing-off conditions are performed by means of the analysis of self-evaluation questionnaires compiled by the patients, which are however subjected to reliability problems tied to the subjective evaluation of the patient himself/herself (e.g. via placebo effect).

It is also known to execute the monitoring by means of wearable electronic devices (of the abovementioned type) provided with accelerometers adapted to detect the movements of the patient so as to carry out an evaluation of the wearing-off block (significant reduction of the movements), of the tremor and of the dyskinesia. Several examples of this device type are described in the U.S. Pat. Nos. 9,393,418 and 9,602,046.

Nevertheless, such devices of known type allow executing an evaluation exclusively centered on the detection and quantification of the motor symptoms, by means of the measurement of the movements of the patient. In particular, such solutions are only employable for monitoring functions, but do not allow obtaining any information usable for identifying symptoms predictive of the wearing-off conditions.

It is also known, from several studies of the biomedical field (for example from the article “Prediction of Freezing of Gait in Parkinson's From Physiological Wearables: An Exploratory Study” by Mazilu et al., IEEE Journal of Biomedical and Health Informatics, Vol. 19, No. 6), to observe the variation of the skin conductance in order to characterize the so-called freezing conditions (or freezing of gait) to which people affected by Parkinson's disease are subjected. In particular, the freezing conditions are manifested in the form of a temporary and uncontrolled incapacity to conduct movements of brief duration (from several seconds to several minutes). Such phenomenon occurs suddenly, in particular while walking, but can also affect the movements for speaking, writing and eye opening.

Nevertheless, the freezing conditions have a clinical nature completely different from that of the aforesaid wearing-off conditions and therefore they do not seem able to provide useful indications for identifying symptom predictive of such wearing-off conditions.

The patent application EP 3076868 describes a device of known type for monitoring the burnout and/or chronic fatigue syndrome stage by means of the detection of the skin conductance. In particular, such device is adapted to calculate characteristics of the skin conductance signal correlated with the signal peaks, such as for example the ascending ramps, the ascending time, the height and the number of the peaks.

The patent application EP 1519679 describes a device of known type for monitoring the autonomous nervous system of a sedated patient. In particular, such device provides for detecting the information relative to the nervous system based on the number and frequency of the signal peaks that have occurred within specific time periods.

Also the latter devices of known type, however, do not allow providing useful information for identifying symptoms predictive of the wearing-off conditions.

### Presentation of the Invention

In this situation, the main object of the present invention is that of overcoming the drawbacks manifested by the solutions of known type, by providing a device for predicting the wearing-off conditions in a patient affected by Parkinson's disease, which is capable of predicting, ahead of time and in a reliable manner, the onset of wearing-off conditions.

Further object of the present invention is to provide a device for predicting the wearing-off conditions in a patient affected by Parkinson's disease, which allows warning further subjects ahead of time regarding the onset of the wearing-off conditions in the patient. Further object of the present invention is to provide a device for predicting the wearing-off conditions in a patient affected by Parkinson's disease, which can be worn in a comfortable and easy manner by the patient, in particular allowing him/her to continue using his/her hands.

Further object of the present invention is to provide a device for predicting the wearing-off conditions in a patient affected by Parkinson's disease, which has a reduced bulk.

### BRIEF DESCRIPTION OF THE DRAWINGS

The technical characteristics of the invention, according to the aforesaid objects, are clearly found in the contends of the below-reported claims and the advantages thereof will be more evident from the following detailed description, made with reference to the enclosed drawings, which represent a merely exemplifying and non-limiting embodiment of the invention, in which:

FIG. 1 shows an illustration of possible positions of the body in which the present device can be worn by a patient;

FIG. 2 an embodiment example of the present device, adapted to be worn on the arm or on the wrist;

FIG. 3 shows a simplified block diagram of the device, object of the present invention;

FIG. 4 shows a graph that reports a first clinical example of a time succession of a physiological parameter (given by the skin conductance), detected by the device, object of the present invention, in a specific time period (of 20 hours);

FIG. 5 shows two graphs relative to the aforesaid first example, of which the upper graph represents the time succession of the skin conductance illustrated in FIG. 4 (with the values of such parameter normalized according to a specific factor and with the tonic component of the skin conductance superimposed), and the second graph represents the measurements detected by the device in the same time period and representative of the movements of the patient;

FIG. 6 shows two graphs relative to a second clinical example, of which the upper graph represents the time succession of the skin conductance in a specific time period of 13 hours (with the values of such parameter normalized according to a specific factor and with the tonic component of the skin conductance superimposed), the second graph represents the measurements detected by the device in the same time period and representative of the movements of the patient.

### DETAILED DESCRIPTION OF A PREFERRED EMBODIMENT

With reference to the enclosed figures, reference number 1 overall indicates the device for predicting the wearing-off conditions in a patient affected by Parkinson's disease, object of the present invention.

The present device 1 is intended to be worn by a user in a specific position of the body and has the object of detecting, a little beforehand (e.g. 10-30 minutes), the onset of a wearing-off condition in the patient.

As is known, the wearing-off condition in a Parkinson's subject is a clinical phase in which the patient is subjected to generalized motor blocks of relatively long duration (in which symptoms can be manifested such as tremors, rigidity or slowness in movements, difficulty in starting the movements, involuntary movements, dyskinesia, dystonia), which also occur in the presence of a well-calibrated pharmacological therapy.

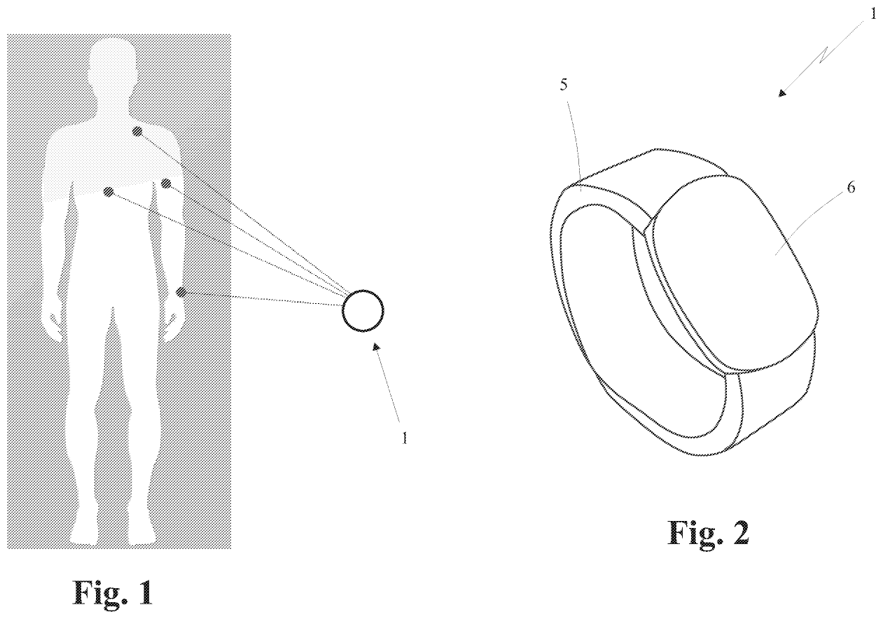

With reference to the application example of FIG. 1, the device 1, object of the present invention, can be arranged for being applied at multiple possible parts of the body of the patient, such as an arm, a wrist, fingers, waist, chest, a shoulder, the neck, etc.).

For example, in the event in which the device 1 is arranged for being applied to the wrist or to the arm, this is configured in bracelet form, intended to be applied around such part of the body.

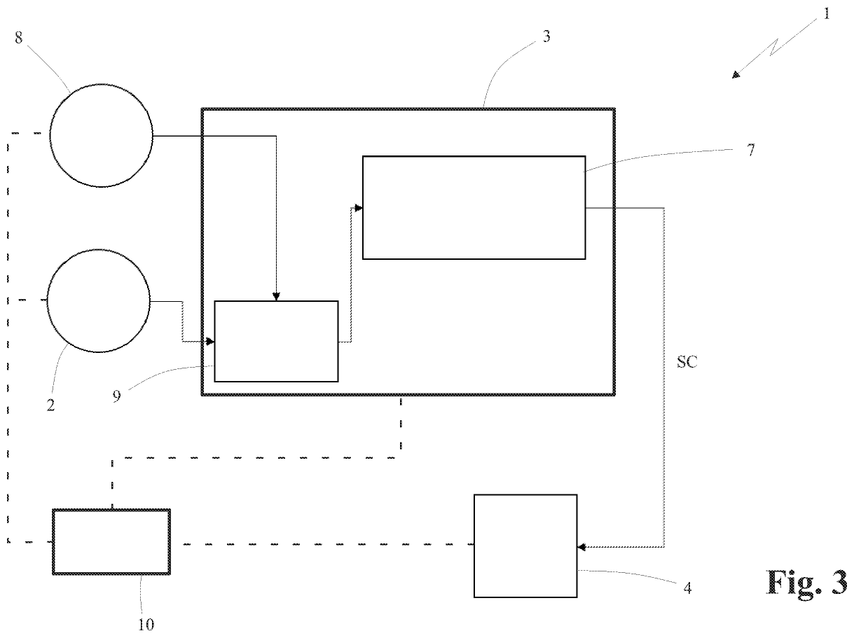

With reference to the exemplifying diagram of FIG. 3, the device 1 comprises an electrodermal activity sensor 2 intended to be placed at the skin of a patient. Such electrodermal activity sensor 2 is arranged for detecting at least one electrical magnitude associated with the skin conductance of the patient himself/herself and for generating corresponding measurement signals (preferably of electric type).

In addition, the device 1 comprises an electronic control unit 3, which is operatively (and preferably electrically) connected to the electrodermal activity sensor 2 in order to receive the aforesaid measurement signals, and is arranged for obtaining, from the latter, a time succession of values of a physiological parameter PF representative of the skin conductance of the skin of the patient.

In particular, such physiological parameter PF represents information tied to the electrodermal activity of the skin of the patient and, advantageously, corresponds with the same skin conductance.

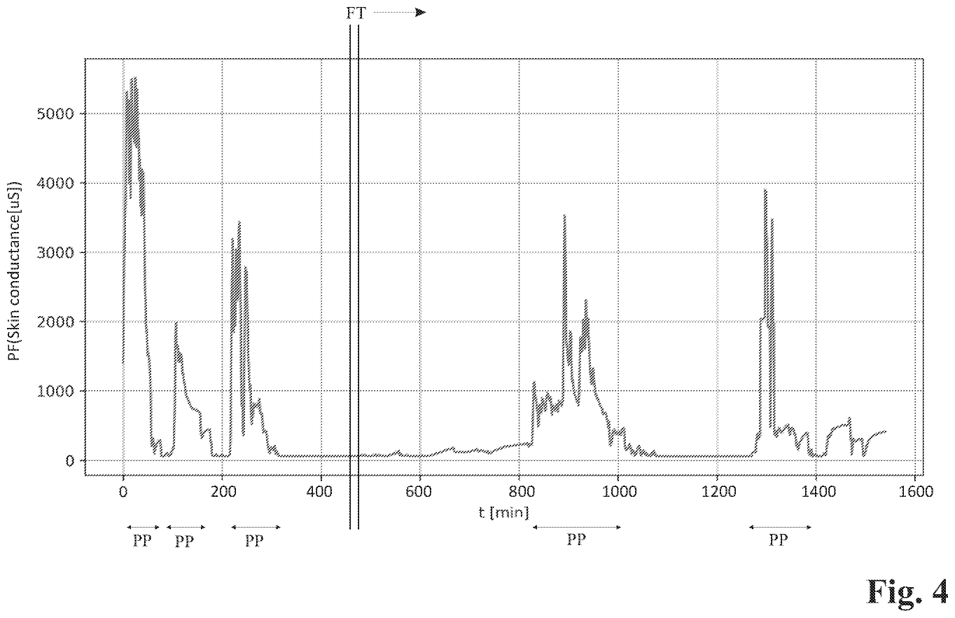

One example of the time succession of the physiological parameter PF obtained by the electronic control unit 3 is illustrated in the graph of FIG. 4 and will be discussed in detail hereinbelow.

The device 1 also comprises a communication unit 4 operatively connected to the electronic control unit 3, which is arranged for controlling on such communication unit 4 to emit an off-notification signal aimed to indicate that a wearing-off condition in the patient is close to being reached.

Advantageously, the device 1 comprises one or more coupling elements 5, preferably of flexible material, and arranged for being constrained to a corresponding part of the body of the patient (such as an arm, a wrist, a finger, the chest, etc.).

For example, the coupling element 5 can be shaped in band or belt form, susceptible of being wound and tightened around the part of the body where it is desired to position the device 1.

In particular, with reference to the embodiment of FIG. 1, the coupling element 5 comprises a flexible band, closed or closeable as a loop, intended to be tightened around the part of the body of the patient, such as the arm or the wrist.

Preferably, the device 1 comprises a support body 6 fixed to the coupling element 5 and arranged for supporting, and advantageously containing, the electrodermal activity sensor 2, the electronic control unit 3 and the communication unit 4.

Advantageously, the electrodermal activity sensor 2 is arranged for being placed in contact with the skin of the patient for detecting an electrical magnitude from which it is possible to derive the electrodermal activity, and in particular the skin conductance. For example, the electrodermal activity sensor 2 comprises two electrodes intended to be placed in contact with the skin in two separate positions, and a direct current voltage generator adapted to apply a predetermined voltage between the two electrodes, in a manner such that an electric current affects the skin zone between the two electrodes. Since the intensity of such electric current depends on the skin resistance (and hence on the skin conductance), by means of the detection of this electric current the electrodermal activity sensor 2 is adapted to detect electrical measurements tied to the skin conductance of the patient.

For example, the electrodermal activity sensor 2 can be obtained with one or more conductometers.

Of course, such electrodermal activity sensor 2 can also be obtained with a resistance sensor, or generally with a sensor of electrical magnitudes capable of detecting an electrical magnitude tied to the skin conductance. In particular, the electrodermal activity sensor 2 could be of capacitive type.

As is known, the electrodermal activity of the skin (in particular the skin conductance) varies as a function of the state of the skin sweat glands, whose functioning is regulated by the autonomous nervous system. In particular, a high activity of the autonomous nervous system corresponds with a high activity of the sweat glands, with a consequent increase of the skin conductance, and vice versa.

Normally, the skin conductance is employed for analyzing the emotional behavior of an individual in different situations.

Surprisingly, it was found that the verification of a wearing-off condition in Parkinson's patients is preceded, at least by several minutes (in particular 10-30 minutes), by a continued, significant increase of the overall progression of the skin conductance. For example, in the graph of FIG. 4, it is possible to identify multiple peaks of the progression of the skin conductance which precede corresponding wearing-off periods of the patient. In particular, such peaks, in their ascending section, have continuous and significant growth of the skin conductivity, which is maintained for a time interval at least on the order of minutes.

It is therefore established that the physiological activation of the autonomous nervous system has a particular variation (detectable from the peaks of the skin conductance) before the wearing-off conditions, hence allowing to be able to predict them with suitable notice (e.g. about 10-30 minutes).

In particular, as is visible also in the example of the graph of FIG. 4, the time succession of the physiological parameter PF (in this case constituted by the skin conductance) comprises peak periods PP, in which the values of the physiological parameter PF increase with respect to base values taken on when the patient is substantially at rest in normal conditions.

It was discovered that, after the ascending section of the peak periods PP (normally after 10-30 minutes), the patient is subjected to a wearing-off condition. Therefore, the aforesaid peak periods PP of the time succession of the physiological parameter PF are due to an increase of the activity of the autonomous nervous system tied to the subsequent wearing-off condition.

In accordance with the idea underlying the present invention, the electronic control unit 3 comprises a processing module 7, which is arranged for defining, in the aforesaid succession of values of the physiological parameter PF, a specific movable time window FT of duration greater than or equal to thirty seconds.

The processing module 7 is also arranged for repeatedly updating such movable time window FT, by making it advance along the succession of values of the physiological parameter PF.

In particular, for each new acquisition of measurement signals by the electrodermal activity sensor 2, and hence the subsequent calculation of new values of physiological parameters PF of the time succession, the processing module 7 is arranged for adding one or more values of the physiological parameter PF at the head of the time window FT and to correspondingly remove one or more values at the tail of the latter, maintaining the same amplitude of the time window FT and making it translate forward along the time succession of values.

For each update of the time window FT, the processing module 7 is arranged for calculating, within the time window FT, an overall time variation of the physiological parameter PF indicative of an increase (intended as positive increase) of the skin conductance within the time window FT itself. The aforesaid overall time variation is calculated by means of the processing of the values of the time succession contained in the time window FT, so as to identify a clear increase of the physiological parameter PF within such time window FT, implementing specific calculation algorithms regarding which several examples will be provided hereinbelow.

In addition, the processing module 7 is arranged for comparing the overall time variation of the physiological parameters with a specific threshold value VS, so as to detect, within the time succession of the physiological parameter PF (in particular the skin conductance), significant increases of the values of such physiological parameter PF, which can be indicative of the verification of a wearing-off condition.

In particular, such significant increases of the physiological parameter PF, discriminated based on the aforesaid threshold value VS, detect peaks of the skin conductance of amplitude and duration such to be an indication of a proximate verification of a wearing-off condition.

When the overall time variation of the values of the physiological parameter PF within the time window FT is greater than the threshold value VS (i.e. when a significant increase is identified), the electronic control unit 3 is configured for sending, to the communication unit 4, a control signal SC adapted to enable the latter to emit the corresponding off-notification signal.

Such off-notification signal hence allows warning, for example a third subject or the patient himself/herself, of a probable proximate wearing-off, hence allowing to suitably act (e.g. with opportune assistance interventions or therapeutic treatment operations).

In particular, as discussed above, the significant increases (indicative of a peak period PP of the time progression of the physiological parameter PF) are discriminated as a function of the amplitude of such increases (greater than the threshold value VS) and of their time duration (determined by the time window FT).

Such parameters (threshold value VS, time window FT) allow identifying continuing variations of the overall progression of the physiological parameter PF with an average slope of such variations sufficiently large to indicate the reaching of peaks of the physiological parameter PF which precede the wearing-off conditions.

In substance, the overall time variation of the physiological parameter PF represents a clear variation, to which the progression of such physiological parameter PF is subjected within the entire time window FT (i.e. from the start to the end of such time window FT).

Advantageously, the overall time variation of the physiological parameter PF is obtained as a ratio between an increase of the values of the physiological parameter PF detected in the time window FT and the time in which such variation occurred, said time being given by the duration of the time window FT.

In particular, the overall time variation of the physiological parameter PF is in substance indicative of the slope that the progression of the physiological parameter PF itself takes on mainly within the time window FT. In substance, if such main slope is greater than the threshold value VS, it is representative of a significant increase of the physiological parameter PF which can precede a wearing-off condition of the patient.

Advantageously, the overall time variation of the physiological parameter PF depends both on the local increases and on the local decreases of the physiological parameter PF which can be verified within the time window FT.

Advantageously, the processing module 7 is arranged for determining the overall time variation of the physiological parameter PF as a function of the time derivative of the physiological parameter PF itself calculated within the time window FT.

In particular, the time derivative of the physiological parameter PF can be calculated by using all or only some of the values of the latter within the time window FT.

Preferably, in accordance with a particular embodiment of the invention, within the aforesaid time window FT, the processing module 7 is adapted to calculate the time variation of the physiological parameter PF as a function of the difference between at least one maximum value and a minimum value of the physiological parameter PF detected within the time window FT.

For example, the overall time variation can be calculated as a difference between the maximum value and the minimum value of the physiological parameter PF present within the time window FT, in relation to the time interval (within the time window FT) that exists between such maximum and minimum values.

In accordance with a different embodiment, the overall time variation is obtained from the average value of the time derivative of the physiological parameter PF within the time window FT.

Advantageously, the time window FT is less than or equal to about five minutes, and preferably substantially comprised between one minute and three minutes, for example about two minutes.

In particular, such values of the time window FT allow identifying time variations of the physiological parameter PF that took place in a substantially continuative manner for a time period sufficiently large to be representative of the verification of a peak period PP of the skin conductance which is a premonition of the verification of a proximate wearing-off condition.

As indicated above, the time variations that take place during the time window FT are selected as significant increases if they have amplitude greater than the aforesaid threshold value VS.

In particular, in the present discussion, the values and the variations of the physiological parameter PF are intended in terms of absolute value, sinch such variations of the physiological parameter PF can be positive or negative, as a function of the particular physiological parameter PF considered (for example considering the skin resistance rather than the skin conductance).

In any case, the time variations of interest of the physiological parameter PF are representative of positive increases of the skin conductance of the patient (i.e. in particular variations in which the overall slope of the progression of the physiological parameter PF is increasing). For example, if the physiological parameter PF coincides with the skin conductance, the time variations will be positive variations. If, however, the physiological parameter PF coincide with the skin resistance, the time variations will be negative variations.

In general, the values taken on by the physiological parameter PF (in particular the skin conductance), both the peak values and the base values, as well as the amplitude and the rapidity (the slope) of the significant increases, can vary from patient to patient. In addition, such values can also vary as a function of the part of the body in which the electrodermal activity sensor 3 is positioned. Therefore, the threshold value VS and the time window FT are arranged as a function of such application factors, in particular based on the measurements of the physiological parameter PF executed for such patient. Advantageously, the threshold value VS is determined as a function of peak values detected in at least one part of the time succession of the values of the physiological parameter PF.

Preferably, the electronic control unit 3 is arranged for being set in a calibration condition, in which it determines the threshold value VS based on the values acquired in a specific calibration time interval IT of the time succession of values of the physiological parameter PF.

For example, the electronic control unit 3 can be set in such calibration condition in an initial phase of its operation, for example after it has been applied for the first time on a specific patient.

Preferably, the calibration time interval IT, in which the electronic control unit 3 operates in the calibration condition, has a duration of several hours, for example 3-6 hours.

In particular, in the calibration condition, the processing module 7 is arranged for detecting the peak values (or maximum values) of the succession of values of the physiological parameter PF that are verified in the aforesaid calibration time interval IT, and to calculate the threshold value as a function VS of such peak values.

More in detail, in the calibration condition, the processing module 7 is arranged for determining at least one rest reference value VRR as a function of the values of the time succession detected outside the peak periods PP, in which the physiological parameter PF remains substantially stably close to a base value (which generally is variable from subject to subject) when the individual is not subjected (in addition to wearing-off conditions) to conditions and/or activities that determine a significant variation of the skin sweating.

For example, the values employed for determining the base value are selected if lower than an upper limit value, and/or if they have variations (derived) lower than a specific discrimination value.

In addition, in the calibration condition, the processing module 7 is arranged for determining a peak reference value VRP as a function of the peak values which are verified in the peak periods PP, i.e. in the periods in which the physiological parameter PF has strong increases with respect to the base value.

In particular, the rest reference value VRR and the peak reference value VRP can be obtained by subjecting the values of the time succession to processing functions (such as filtering, means, etc.) adapted to remove insignificant values, for example due to noise, disturbances, spurious oscillations, etc.

For example, the rest reference value VRR is obtained as mean of the values of the physiological parameter PF outside the PP periods, excluding possible further strong deviations from the base value due to possible factors (usually irregular) not tied to the wearing-off condition.

For example, the peak reference value VRP is obtained as the means of the maximum values reached in multiple peak periods.

Advantageously, the processing module 7 is adapted to calculate the threshold value VS as a function of the difference between the rest reference value VRR and the peak reference value VPR (and in particular of the absolute value of such difference). For example, the aforesaid difference between the rest reference value VRR and the peak reference value VPR (and hence advantageously the threshold value VS) is at least on the order of microsiemens, and in particular is comprised between the order of microsiemens and the order of tens of microsiemens.

Suitable, the threshold value VS is proportional to the aforesaid difference (by a specific fixed or variable factor), and is preferably lower than such difference.

In particular, the threshold value VS is comprised between about ⅓ and 1/7 of the difference between the rest reference value VRR and the peak reference value VRP. For example, the threshold value VS can be obtained as:

VS=(VRP−VRR)/5.

Otherwise, the threshold value VS, for example before executing the calibration condition, can be set based on different criteria, e.g. obtained from values retrieved from the literature on the subject.

Advantageously, the electronic control unit 3 of the device 1 comprises an electronic processor (preferably a microprocessor), which is suitably programmed for carrying out the above-described functions.

Preferably, the processing module 7 of the electronic control unit 3 is integrated in the aforesaid electronic processor, being implemented, for example with a particular program installed in such electronic processor. Of course, as an alternative, the processing module 7 can also be implemented with a hardware component different from the aforesaid electronic processor.

Advantageously, the communication unit 4 of the device 1 comprises a wireless communication module (such as a radio transmitter), capable of sending the off-notification signal to a remote unit adapted to receive such signal and arrange warning signals for third parties. Such remote unit, for example, can comprise a portable device (such as a smartphone, a people-finder, a suitable receiver, etc.), a computer of a control station, or in general an electronic device that can be available for a person, such as a medical operator, a nurse, a relative, etc., in a manner such that such person can take suitable provisions in view of a probable proximate wearing-off condition of the patient. Preferably, in accordance with a particular embodiment, the device 1 comprises a signaling unit connected to the communication unit 4 (possibly also integrated in the latter) which, following the reception of the off-notification signal, is capable of emitting a sound, visual or haptic signal in order to warn the patient himself/herself or people nearby.

In accordance with a further particular embodiment, the communication device 4 is arranged for sending a signal to an apparatus adapted to adjust the administration of one or more drugs to the patient according to a specific pharmaceutical therapy.

In accordance for example with the embodiment illustrated in FIG. 2, the processing module 7 and the communication unit 4 are mounted locally, for example on the support body 6 of the device 1.

In accordance with an alternative embodiment, the processing module 7 and the communication unit 4 are mounted remotely, for example in a remote processor (such as a server), and the device 1 comprises a transmitter connected to the electronic control unit 3 adapted to transmit, to the remote processor, the electrical measurements and/or the succession of values of the physiological parameter PF, in a manner such that the processing module 7 and the communication unit 4 can complete their functions, as described above, remotely.

Preferably, the hardware and software components of the device 1 employed for detecting the values of the physiological parameter PF during the aforesaid calibration condition and during the subsequent operation are placed in the same support body 6. Otherwise, the hardware and software components of the device 1 employed for detecting the values of the physiological parameter PF during the calibration condition are arranged on a separate unit of the device 1.

Advantageously, the device 1 comprises at least one inertial sensor 8 intended to be applied to the patient and adapted to measure kinematic variables of the body of the patient himself/herself so as to generate corresponding detection signals.

Preferably, the inertial sensor 8 can be mounted on the support body 6 of the device 1 and can comprise for example an accelerometer or a gyroscope.

The inertial sensor 8 is operatively connected to the electronic control unit 3 in order to send, to the latter, the aforesaid detection signals, from which the electronic control unit 3 is capable of obtaining information relative to the frequency, to the intensity and to the type of movements carried out by the patient.

Such information can be advantageously correlated with the time succession of the physiological parameter PF in order to identify specific interactions between such parameters.

For example, the graph of FIG. 4 shows a test example of a time succession of the physiological parameter PF (corresponding, in this case, to the skin conductance) obtained based on the measurements detected by the electrodermal activity sensor 3 during a time period of 20 hours.

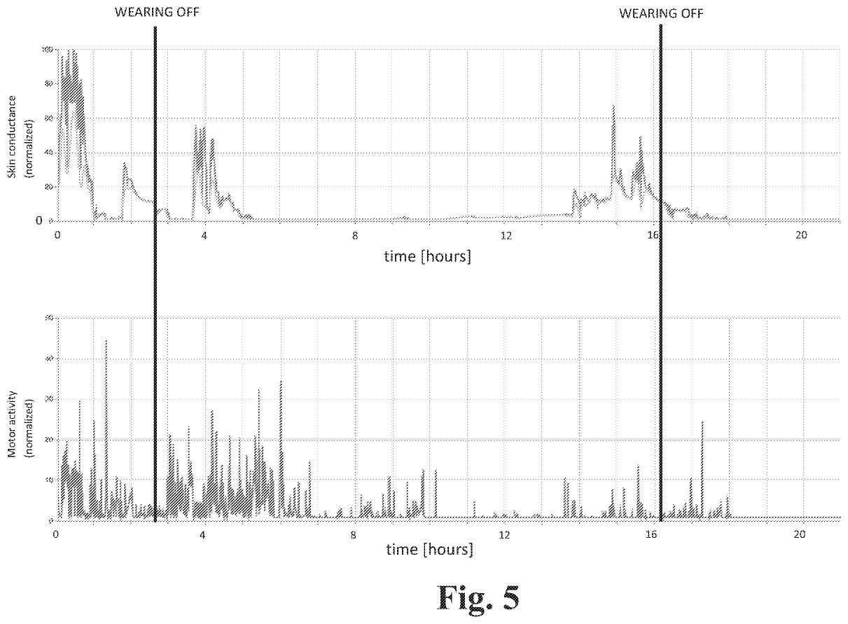

The same graph is reported (with normalized values of the skin conductance) in the upper part of FIG. 5, while in the lower part of such FIG. 5 a further graph is reported which represents the time progression of the measurements (also in this case normalized) detected by the inertial sensor 3 in the same aforesaid time period and representative of the frequency and intensity of the movements completed by the patient.

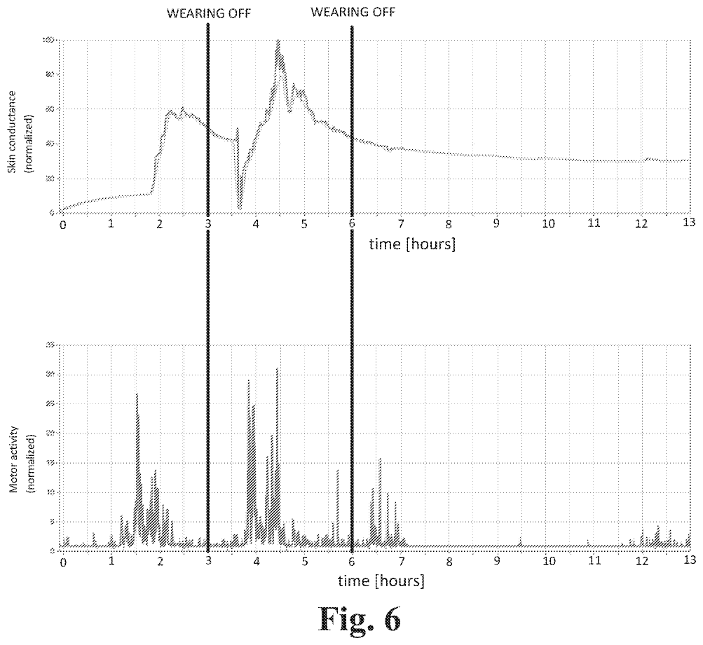

The graphs of FIG. 6 show the aforesaid information retrieved in a different patient in a second clinical example.

Also indicated in such graphs, by means of vertical lines, are the moments in which the wearing-off conditions were encountered in the patient.

As can be detected, the wearing-off conditions were verified following the increase of the values of the physiological parameter PF of corresponding peak periods PP, in particular during the descending phase of such peak periods PP.

In addition, as can be established by the lower graphs of FIGS. 5 and 6, at the wearing-off moments, the measurements detected by the inertial sensor 8 have substantially bottom values, lacking peaks representative of movements by the patient, thus demonstrating the absence of substantial movements by the patient consistent with the existing wearing-off condition.

Advantageously, the electronic control unit 3 comprises a conditioning module for the signal 9 connected to the electrical sensor 3 and preferably to the inertial sensor 8, in order to receive, respectively, the measurement signals and the detection signals generated by such sensors 3, 8. Such conditioning module 9 is arranged for processing such measurement/detection signals so as, in particular, to eliminate spurious components (due for example to noise, disturbances, etc.) by means of specific processes for processing and/or transforming the signal, such as operations of filtering, interpolation, smoothing, etc.

The conditioning module 9 is connected to the electronic control unit 3 in order to send, to the latter, the measurement/detection signals conditioned in a manner such to facilitate a correct processing of the data contained therein by the electronic control unit 3 itself.

Advantageously, the device 1 comprises an electrical power supply unit 10, such as for example a battery, electrically connected to the electronic control unit 3, to the electrodermal activity sensor 2, to the communication unit 4 and, preferably, to the inertial sensor 8 so as to provide to the latter the electrical energy (preferably in direct current) suitable for the operation thereof. Such electrical power supply unit 10 is for example placed within the support body 6 of the device 1.

Hereinbelow, a method is described for operating the device 1 of the abovementioned type.

Hereinbelow, for the sake of description simplicity, reference will be made to the same nomenclature introduced up to now, even if it must be intended that the present method can be applied to adjustment devices that are provided with all the above-considered characteristics.

Such operating method provides that the electrodermal activity sensor 2 be applied to the skin of a patient, in particular by making the patient himself/herself wear the device 1. Preferably, the electrodermal activity sensor 2, and in particular its electrodes, is placed in contact with the skin.

The electrodermal activity sensor 2 is actuated for detecting at least one electrical magnitude associated with the skin conductance of the patient, in a manner such to generate the corresponding measurement signals that are sent to the electronic control unit 3.

The latter, therefore, receives the measurement signals from the electrodermal activity sensor 2 and obtains therefrom a time succession of values of a physiological parameter PF representative of the skin conductance of the skin of the patient.

The electronic control unit 3, as a function of the values of the physiological parameter PF, controls the communication unit 4 to emit at least one off-notification signal indicative of the proximate verification of a wearing-off condition of the patient.

The operating method provides that the processing module 7 of the electronic control unit 3 defines, in the time succession of values of the physiological parameter PF, a specific movable time window FT of duration greater than or equal to thirty seconds, preferably lower than about five minutes and, in particular, comprised between about one minute and three minutes.

The updating module, following the acquisition of new measurement signals by the electrodermal activity sensor 2 (and hence the obtainment of new corresponding values of the physiological parameter PF), repeatedly executes an updating operation of the time window FT by making it advance along the time succession of values acquired of the physiological parameter PF, in accordance with that already discussed above. With every updating operation of the time window FT, the processing module 7 calculates, within the time window FT itself, an overall time variation of the physiological parameter PF indicative of an increase of the skin conductance within the time window FT. Such calculation is obtained by means of the processing of the values of the time succession contained in the time window FT.

Advantageously, the aforesaid time variation of the physiological parameter PF is obtained by means of the time derivative of the physiological parameter PF within the time window FT, in particular according to that discussed above on the matter.

According to the present method, the processing module 7 compares the overall time variation of the physiological parameter PF with a specific threshold value VS and, if the overall time variation is greater than such threshold value VS, sends the communication unit 4 a control signal SC, which enables the latter to emit the off-notification signal, allowing to warn the patient or third parties of a proximate probable wearing-off condition (as discussed above).

Advantageously, the method for operating the device 1 provides for a calibration step, preferably executed when the device 1 is initially applied to the patient so as to set the value of the threshold value VS.

More in detail, in such calibration step, the electronic control unit 3 is set, for example by means of a suitable control enabled by the user, in the aforesaid calibration condition, in which the electronic control unit 3 determines the threshold value VS based on the values acquired in a specific calibration time interval IT of the time succession of values of the physiological parameter PF, in accordance with that discussed above.

The invention thus conceived therefore attains the pre-established objects.

1. A device for predicting the wearing-off conditions in a patient affected by Parkinson's disease, said device comprising:
an electrodermal activity sensor (2) intended to be placed at the skin of a patient and arranged for detecting at least one electrical magnitude associated with the skin conductance of the patient and to generate corresponding measurement signals;
an electronic control unit (3), which is operatively connected to said electrodermal activity sensor (2) in order to receive said measurement signals, and is arranged for obtaining, from said measurement signals, a time succession of values of a physiological parameter (PF) representative of the skin conductance of the skin of the patient;
a communication unit (4) operatively connected to said electronic control unit (3) which is arranged for controlling said communication unit (4) to emit at least one off-notification signal;
wherein said electronic control unit (3) comprises at least one processing module (7), which is arranged for
defining, in said time succession of values of said physiological parameter (PF), a movable time window (FT) of duration greater than or equal to thirty seconds, and
updating said movable time window (FT) by making it advance along said time succession of values;
wherein, for each update of said movable time window (FT), said processing module (7) is arranged for:
calculating, within said movable time window (FT), an overall time variation of said physiological parameter (PF) indicative of an increase of the skin conductance within said movable time window (FT), by means of the processing of the values of said time succession contained in said movable time window (FT);
comparing said overall time variation with a specific threshold value (VS);
with said overall time variation greater than said threshold value (VS), sending to said communication unit (4) at least one control signal (SC), which is adapted to enable said communication unit (4) to emit said off-notification signal.

2. The device of claim 1, wherein said processing module (7) is arranged for determining said overall time variation as a function of the time derivative of said physiological parameter (PF) in said movable time window (FT).

3. The device of claim 1, wherein said processing module (7) is arranged for determining said overall time variation as a function of the average value of the time derivative of said physiological parameter (PF) calculated within said movable time window (FT).

4. The device of claim 1, wherein said movable time window (FT) is less than or equal to about five minutes.

5. The device of claim 4, wherein said movable time window (FT) is substantially comprised between one minute and three minutes.

6. The device of claim 1, wherein said processing module (7) is arranged for determining said time variation as a function of the difference between at least a maximum value and a minimum value of said physiological parameter (PF) in said movable time window (FT).

7. The device of claim 1, wherein said threshold value (VS) is determined as a function of peak values of said time succession of values of said physiological parameter (PF).

8. The device of claim 7, wherein said electronic control unit (3) is arranged for being set in a calibration condition, wherein, in a specific calibration time interval (IT), said processing module (7) is arranged for detecting said peak values of said time succession of values, and for calculating said threshold value (VS) as a function of said peak values.

9. The device of claim 8, wherein said threshold value (VS) is a function of at least one average value of said peak values detected in said calibration time interval (IT).

10. The device of claim 8, wherein said time succession comprises peak periods (PP), in which one or more of said peak values are contained,
wherein, in said calibration condition, said processing module (7) is arranged for determining at least one rest reference value (VRR) as a function of the values of said time succession outside said peak periods (PP), and at least one peak reference value (VRP) as a function of said peak values, and for calculating said threshold value (VS) as a function of the difference between said peak reference value (VRP) and said rest reference value (VRR).

11. The device of claim 10, wherein that said threshold value (VS) is comprised between about ⅓ and 1/7 of the difference between said rest reference value (VRR) and said peak reference value (VRP).

12. The device of claim 1, wherein said overall time variation of said physiological parameter (PF) is obtained as a ratio between an increase of the values of said physiological parameter (PF) detected in said movable time window (FT) and the duration of said movable time window (FT).

13. The device of claim 1, wherein the overall time variation of said physiological parameter (PF) is indicative of the slope that the progression of said physiological parameter (PF) takes on mainly within the entire said movable time window (FT).
